(19) United States
(12) Patent Application Publication
(10) Pub. No.: US 2019/0307728 A1
(43) Pub. Date: Oct. 10, 2019

(54) METHODS OF TREATMENT WITH A 2,4,5-TRISUBSTITUTED 1,2,4-TRIAZOLONE

(71) Applicants: Bayer Pharma Aktiengesellschaft, Berlin (DE); The Broad Institute, Inc., Cambridge, MA (US)

(72) Inventors: Andreas Janzer, Berlin (DE); Steven James Ferrara, Cambridge (US)

(21) Appl. No.: 16/367,708

(22) Filed: Mar. 28, 2019

Related U.S. Application Data
(60) Provisional application No. 62/655,355, filed on Apr. 10, 2018.

(51) Int. Cl. A61K 31/4196; A61K 45/06; A61P 35/00

(52) U.S. Cl. CPC A61K 31/4196; A61P 35/00; A61K 45/06

(57) ABSTRACT

The present invention provides a method of treating a mutant IDH cancer in a subject comprising administering to a subject in need thereof a therapeutically effective amount of Compound (I):

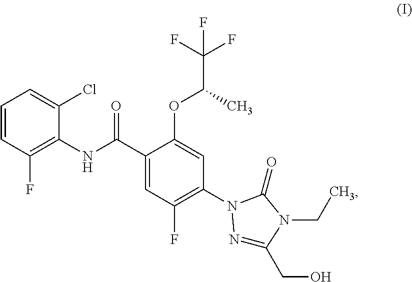 [SMILES: CCn1c(CO)nn(-c2cc(O[C@@H](C)C(F)(F)F)c(C(=O)Nc3c(F)cccc3Cl)cc2F)c1=O]

and the use of said compound for manufacturing pharmaceutical compositions for the treatment or prophylaxis of diseases, in particular of hyperproliferative disorders bearing an IDH mutation, as a sole agent or in combination with other active ingredients.

### CROSS REFERENCE TO RELATED APPLICATIONS

This application claims priority to U.S. Provisional Application No. 62/655,355, filed 10 Apr. 2018, the disclosure of which is incorporated herein in its entirety.

The present invention provides methods of treatment of hyperproliferative and/or inflammatory disorders, particularly cancer bearing an IDH mutation, comprising administering a 2,4,5-trisubstituted 1,2,4-triazolone compound which is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide. as a sole agent or in combination with other active ingredients to a subject in need thereof.

### BACKGROUND OF THE DISCLOSURE

Acute myeloid leukemia (AML) is the most common acute leukemia in humans with a 5 year survival of only about 30%. AML is a malignancy of the myeloid line of blood cells. The incidence rates and chances of cure are highly age dependent. The chemotherapy standard of care for AML has not changed significantly over the last decades highlighting the need for novel therapies. A major hallmark of AML is differentiation arrest of the leukemic cells at early stages of cellular differentiation. The potential of leukemic differentiation therapy can be seen with the success of ATRA or arsenic trioxide inducing differentiation in acute promyelocytic leukemia (APL). Around 10% of AML belong to the APL subtype where leukemic cells are harbouring a chromosomal translocation resulting in fusions of oncoproteins involving the retinoic acid receptor. While treatment with ATRA or arsenic trioxide leads to a dramatic increase of patient survival, with overall survival rates of over 70%, unfortunately a comparable differentiation therapy for the non-APL AMLs is lacking (Management of acute promyelocytic leukemia: recommendations from an expert panel on behalf of the European LeukemiaNet, Sanz M. A. et al, Blood 2009, 113(9), 1875-1891). Therefore new therapies inducing differentiation of AML cells are of high interest and medical need. IDH mutations in AML cancer cells occur with about 7% in IDH1 and about 11% in IDH2.

Dihydroorotate Dehydrogenase (DHODH)

DHODH is located in the mitochondria and the enzyme responsible for the 4^{th} and rate limiting step in de novo pyrimidine synthesis converting dihydroorotate to orotate (Dihydroorotat-ubiquinone oxidoreductase links mitochondria in the biosynthesis of pyrimidine nucleotides, Löffler M. et al, Molecular and Cellular Biochemistry 1997, 174, 125-129).

As pyrimdine production is essential for DNA and RNA synthesis DHODH is highly important for cellular proliferation. The enzyme is considered an attractive drug target for cancer, immunological, parasitic and viral diseases and DHODH small molecule inhibitors like Leflunomide/Teriflunomide and Brequinar have been approved for clinical use in Rheumatoid Arthritis and Multiple Sclerosis. Additionally, preclinical studies indicate that DHODH inhibitors may be useful for the treatment of haematological cancer indications, for the treatment of solid tumors (e.g., neuroblastoma, melanoma, colon, breast and lung tumors), for the treatment of parasitic diseases (e.g., malaria), and for viral disease therapy.

Efforts to identify new therapeutic targets to overcome myeloid differentiation blockade have been largely unsuccessful. Small molecule inhibitors of mutant isocitrate dehydrogenase (IDH)2 (Wang et al., 2013, Science 340, 622-626) or IDH1 (Okoye-Okafor et al., 2015, Biol. 11, 878-886) may be capable of inducing cellular differentiation among that subset (15%) of patients with IDH1/2 mutations. However, the remainder of AML cases involve complex and heterogeneous combinations of chromosomal alterations and gene mutations (Cancer Genome Atlas Research Network, 2013, N. Engl. J. Med. 368,2059-2074), highlighting the difficulty in developing mutation-specific therapies.

Isocitrate Dehydrogenase (IDH)

The enzyme isocitrate dehydrogenase (IDH) normally catalyzes the oxidation process of isocitrate to obtain alpha-ketoglutarate after decarboxylation wiothin the citric acid cycle. There exist three isoforms of IDH in humans, IDH1, IDH2 and IDH3. IDH3 is located in the mitochodria only and catalyzes the reaction as well as the conversion of NAD⁺ to NADH+H⁺ as part of the citric cycle whereas IDH1 and IDH2 are located in the cytosol, in mitochondria and peroxisomes and catalyze the reaction mentioned above outside the citric acid cycle converting NADP⁺ into NADPH+H⁺.

From GeneBank NCBI a human gene sequence of the corresponding mRNA of IDH1 is known under NM_005896.3, a human gene sequence of the corresponding mRNA of IDH2 is known under NM_002168.3. For IDH3 there exist three human gene sequences reflecting the three different subunits alpha, beta and gamma, whereby the alpha subunit exists twice in the complete IDH3, a human gene sequence of the corresponding mRNA of IDH3 alpha (IDHA) is known under NM_005530, a human gene sequence of the corresponding mRNA of IDH3 beta (IDH3B) is known under NM_174855 and a human gene sequence of the corresponding mRNA of IDH3 gamma (IDHG) is known under NM_174869.

The respective human proteins corresponding to the gene sequences mentioned above are also available from GeneBank NCBI for IDH1: NP_005887.2, for IDH2: NP_002159.2, for IDH3A: NP_005521.1, for IDH3B: NP_777280.1 and for IDH3C: NP_777358.1 respectively.

From Dang et al, Nature 2009, Dec. 10; 462(7274):739.744 it is known that mutations in IDH1 associated with cancer result in a different function of IDH due to structural changes. Mutated IDH1 bear a new ability to catalyze the NADPH/H⁺ dependent reduction of alpha-ketoglutarate to R(−)-2-hydroxyglutarate (2HG). Especially a mutations where Arginine at position 132 is being replaced by histidine R132H, cysteine R132C, glycine R132G, leucine R132L or serine R132S is mentioned. Other point mutations have been found at key positions of the catalytic domain IDH1R100, IDH1R132, IDH1G97 e.g. G97D, IDH2R140 e.g. R140L, R140W, R1400 and IDH2R172, e.g. R172G or R172K. The arginine mutation R132H seems to correlate with an elevated risk of a patient having such a mutation to develop brain cancer.

Furthermore Lu et al reported in Nature 2012, 483 (7390):474-478 that recurrent mutations of IDH1 and IDH2 have been identified in gliomas, acute myeloid leukemias and chondrosarcomas sharing a novel enzymatic property of producing 2-hydroxyglutarate from alpha-ketoglutarate. Producing of 2-hydroxyglutarate prevents histone demethylation leading to a block of cell differentiation.

From WO 2015/121210 mutant IDH inhibitors are known.

U.S. Pat. No. 6,444,613 B1 relates to the field of defoliants, in particular thidiazuron-comprising mixtures, and their use in crops of cotton. These mixtures comprise among others 2,4,5-trisubstituted 1,2,4-triazolone compounds as herbicides, which inhibit the enzyme protoporphyrinogen-(IX) oxidase (PPO inhibitors).

WO199802422 describes substituted aromatic carbonyl compounds, among others 2,4,5-trisubstituted 1,2,4-triazolone compounds, as herbicides.

From CN106543139 some triazolone compounds are known as agrochemicals.

US 2016/0251341 A1 describes triazole compounds as serine protease inhibitors useful for the inhibition of thrombin and/or kallikrein.

WO2010/077686 A1 describes sirtuin-modulating compounds, e.g. isoindolinone and related compounds, and methods of use thereof. The sirtuin-modulating compounds may be used for increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing as well as diseases or disorders that would benefit from increased mitochondrial activity.

WO 2013/186692 A1 describes triazolone compounds as mPGES-1 inhibitors, useful in the treatment of pain and/or inflammation from a variety of diseases or conditions, such as asthma, osteoarthritis, rheumatoid arthritis, acute or chronic pain and neurodegenerative diseases.

WO2017117372 disclose a method of treating a mutant IDH cancer in a subject by administering an antimetabolite or a DHODH inhibitor.

However, the state of the art does not describe the method of treatment as disclosed herein with N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide.

### DESCRIPTION

In a first aspect the invention provides a method for treatment of hyperproliferative and/or inflammatory disorders, such as cancer, especially where the cancer cell bears an IDH mutation, comprising administering an effective amount of Compound (I)

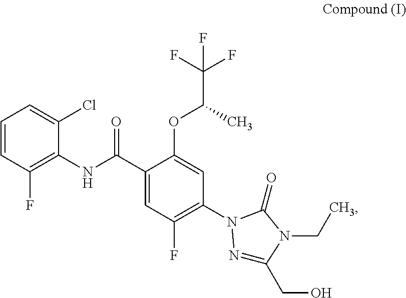 [SMILES: CCn1c(CO)nn(-c2cc(O[C@@H](C)C(F)(F)F)c(C(=O)Nc3c(F)cccc3Cl)cc2F)c1=O]

or a tautomer, an N-oxide, a salt, a salt of a tautomer or a salt of an N-oxid thereof to a patient in need thereof.

In a further aspect the invention provides Compound (I) for use in a method of treating a mutant IDH cancer in a subject comprising administering to a subject in need thereof a therapeutically effective amount of said Compound (I).

In another aspect the invention provides the use of the Compound (I) for the treatment of hyperproliferative diseases comprising administering Compound (I) to a patient in need thereof suffering from a cancer type characterized by having an IDH mutation.

In particular, Compound (I) of the present invention effectively inhibits DHODH and may additionally inhibit cancer cell growth of cancer cells bearing an IDH mutation and may therefore be used for the treatment or prophylaxis of hyperproliferative and/or inflammatory disorders, such as cancer, especially cancer characterized by an IDH mutation.

In a further aspect the invention provides a method as mentioned above where the mutated IDH is R132C, R132G, R132L, R132S, G97D, R140L, R140W, R140Q, R172G and R172K.

### DETAILED DESCRIPTION

Definitions

As used herein, the term “as defined supra” means as defined anywhere in the specification and claims.

By “subject” is meant a mammal, including, but not limited to, a human or non-human mammal, such as a bovine, equine, canine, ovine, rodent, or feline.

It is possible for Compounds (I) to exist as isotopic variants. The invention therefore includes one or more isotopic variant(s) of said Compound (I), particularly deuterium-containing Compounds (I) in the methods defined supra.

The term “mutant IDH inhibitor” includes any IDH inhibitor inhibiting a cancer type having an IDH mutation as described herein, more especially those as described in WO 2015/121210.

The term “Isotopic variant” of a compound or a reagent is defined as a compound exhibiting an unnatural proportion of one or more of the isotopes that constitute such a compound.

The term “Isotopic variant of Compound (I)” is defined as Compound (I) exhibiting an unnatural proportion of one or more of the isotopes that constitute such a compound.

The terms “related to IDH mutation” or “mutated IDH” or bearing an IDH mutation” or “characterized by an IDH mutation” are all used synonymously including the mutations as defined supra.

The term “potentially bearing an IDH mutation” means that before deciding on treatment it might be unclear whether or not the cell or cancer type is characterized by an IDH mutation e.g. because it was still scientifically not known, or has not yet been determined nor analyzed.

The expression “unnatural proportion” in relation to an isotope means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of Compound (I) preferably contain deuterium (“deuterium-containing Compound (I)”). Isotopic variants of Compound formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into Compound (I). These isotopic variants of Compound (I) are useful for in vivo imaging applications.

Deuterium-containing and ¹³C-containing Compound (I) can be used in mass spectrometry analyses in the context of preclinical or clinical studies.

Isotopic variants of Compound (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing Compound (I)” is defined as Compound (I), in which one or more hydrogen atom(s) is/are replaced by one or more deuterium atom(s) and in which the abundance of deuterium at each deuterated position of Compound (I) is higher than the natural abundance of deuterium, which is about 0.015%. Particularly, in a deuterium-containing Compound (I) the abundance of deuterium at each deuterated position of Compound (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into Compound (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing Compound (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

Compound (I) may have multiple potential sites of attack for metabolism. To optimize the above-described effects on physicochemical properties and metabolic profile, deuterium-containing Compound (I) having a certain pattern of one or more deuterium-hydrogen exchange(s) can be selected. Particularly, the deuterium atom(s) of deuterium-containing Compound (I) is/are attached to a carbon atom and/or is/are located at those positions of the Compound (I), which are sites of attack for metabolizing enzymes such as e.g. cytochrome P₄₅₀.

In another embodiment the method of present invention includes a deuterium-containing Compound of (I) having 1, 2, 3 or 4 deuterium atoms, particularly with 1, 2 or 3 deuterium atoms.

By “stable compound' or “stable structure” is meant a compound that is sufficiently robust to survive isolation to a useful degree of purity from a reaction mixture, and formulation into an efficacious therapeutic agent.

Further, Compound (I) can exist as N-oxides, which are defined in that at least one nitrogen of compound (I) is oxidised. The method of the present invention includes all such possible N-oxides.

Compound (I) also may exist as hydrate, solvate, prodrug, salt, in particular pharmaceutically acceptable salt, and/or co-precipitates.

Compound (I) may exist as a hydrate, or as a solvate, wherein said compound contains polar solvents, in particular water, methanol or ethanol for example, as structural element of the crystal lattice of the compound. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The method of the present invention includes all such hydrates or solvates.

Further, it is possible for Compound (I) to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the Compound (I).

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of Compound (I) may be, for example, an acid-addition salt bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of Compound (I) which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of Compound (I) to be prepared by reaction said compound with the appropriate inorganic or organic acid via any of a number of known methods. Alternatively, alkali and alkaline earth metal salts are prepared by reacting Compound (I) with the appropriate base via a variety of known methods.

The method of the present invention includes all possible salts of Compound (I) as single salts, or as any mixture of said salts, in any ratio.

The method of the present invention includes diastereomers, racemates, tautomers, N-oxides, hydrates, solvates, and salts of Compound (I), and mixtures of same.

In the present text, in particular in the Experimental Section, for the synthesis of intermediates and of Compound (I), when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na+”, for example, mean a salt form, the stoichiometry of which salt form not being specified.

This applies analogously to cases in which synthesis intermediates or example Compound (I) or salts thereof have been obtained, by the preparation and/or purification processes described, as solvates, such as hydrates, with (if defined) unknown stoichiometric composition.

Furthermore, the method of the present invention includes use of all possible crystalline forms, or polymorphs, of Compound (I), either as single polymorph, or as a mixture of more than one polymorph, in any ratio.

Moreover, the method of the present invention also includes prodrugs of Compound (I). The term “prodrugs” here designates compounds which themselves can be biologically active or inactive, but are converted (for example metabolically or hydrolytically) into Compound (I) during their residence time in the body.

In accordance with certain embodiments, the present invention provides a method for treatment of hyperproliferative and/or inflammatory disorders, such as cancer, especially where the cancer cell bears an IDH mutation, comprising administering an effective amount of Compound (I), supra, or a tautomer, an N-oxide, a salt, a salt of a tautomer or a salt of an N-oxide thereof to a patient in need thereof.

In another embodiment, the present invention provides a method for treatment of hyperproliferative and/or inflammatory disorders, such as cancer, especially where the cancer cell bears an IDH mutation, comprising administering an effective amount of Compound (I), supra, which is N-(2-chloro-6-fluorophenyl)-4-[4-ethyl-3-(hydroxym ethyl)-5-oxo-4,5-dihydro-1H-1,2,4-triazol-1-yl]-5-fluoro-2-{[(2S)-1,1,1-trifluoropropan-2-yl]oxy}benzamide, or a tautomer, an N-oxide, a salt, a salt a tautomer or a salt of an N-oxide thereof to a patient in need thereof.

The method of the present invention provides Compound (I) which is disclosed in the Example Section, infra.

Compound (I) can be prepared according to the method as disclosed in the example section.

Compound (I) demonstrates a valuable pharmacological spectrum of action. Compound of effectively inhibits DHODH, may inhibit mutated IDH and it is possible therefore that said compound is used for the treatment or prophylaxis of diseases, preferably hyperproliferative and/or inflammatory disorders in humans and animals.

Thus it may be possible to use Compound (I) for the treatment of cancer types characterized by an IDH mutation.

Compound (I) can be utilized in a method to inhibit the activity of DHODH. This method comprises administering to a mammal in need thereof, including a human, an effective amount of Compound (I), or a pharmaceutically acceptable salt, polymorph, hydrate, solvate or ester thereof, for the treatment of hyperproliferative and/or inflammatory disorders.

Another aspect of the invention is a method of inhibiting proliferation of a cell bearing an IDH mutation (a mutant IDH cancer), comprising contacting the cell with Compound (I).

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include sarcomas, and haematological malignancies including but not limited to leukemias, lymphomas, multiple myeolomas.

One aspect of the invention is the use of Compound (I) for the treatment of cancer, especially a mutant IDH cancer, Compound (I) for use in the treatment of cancer bearing an IDH mutation as well as a method of treatment of mutant IDH cancer diseases, comprising administering a specific amount of Compound (I).

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, gliosarcoma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt's lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma and Sezary syndrome.

Sarcomas include, but are not limited to, sarcoma of the soft tissue, gliosarcoma, osteosarcoma, malignant fibrous histiocytoma, lymphosarcoma, and rhabdomyosarcoma.

Leukemias include, but are not limited to acute lymphoblastic leukemia, acute myeloid leukemia, (acute) T-cell leukemia, acute lymphoblastic leukemia, acute lymphocytic leukemia (ALL), acute monocytic leukemia (AML), acute promyelocytic leukemia (APL), bisphenotypic B myelomonocytic leukemia, chronic lymphocytic leukemia, chronic myelogenous leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia.

Inhibition of DHODH can also lead to differentiation of tumor initiating cells in hematological and solid cancers, especially leukemias.

Another aspect of the invention is a method for controlling cancer (e.g., through threatment, prophylaxis, etc.) characterized by an IDH mutation in a subject (e.g., human, rat, etc.) by administering an effective amount of Compound (I), or a pharmaceutically acceptable salt, polymorph, metabolite, hydrate, solvate or ester thereof to the subject.

In some embodiments, the subject may be administered a medicament, comprising Compound (I) and one or more pharmaceutically acceptable carriers, excipients and/or diluents.

In some embodiments, the method of treatment and/or prophylaxis of a hyperproliferative disorder related to IDH mutation in a subject may comprise administering to the subject an effective amount of Compound (I). The hyperproliferative disorder may be, for example, cancer (the cancer types as defined supra, more particularly leukemia, lymphoma, solid tumors, such as e.g. colorectal carcinoma, lung cancer, ovarian cancer, pancreatic cancer, renal cancer, even more particularly e.g. acute myeloid leukemia, colorectal carcinoma, leukemia, lung cancer, lymphoma, multiple myeloma, ovarian cancer, pancreatic cancer and renal cell carcinoma).

In some embodiments, the method of treatment and/or prophylaxis of a hyperproliferative disorder related to IDH mutation in a subject may comprise administering to the subject an effective amount of Compound (I). The hyperproliferative disorder may be, for example, cancer (e.g., lung cancer, acute myeloid leukemia, acute promyelocytic leukemia (APL), mixed-lineage leukemia (MLL), chronic myeloid leukemia (CML), myelodysplastic syndrome (MDS), lymphoma, glioblastoma, prostate cancer, or any other cancer indication as defined herein).

In another aspect, the present invention provides the use of Compound (I) of the present invention, or a pharmaceutically acceptable salt, polymorph, hydrate, solvate or ester thereof, for the treatment of cancer bearing an IDH mutation, which cancer is selected from acute myeloid leukemia, colorectal carcinoma, leukemia, lung cancer, lymphoma, multiple myeloma, ovarian cancer, pancreatic cancer and renal cell carcinoma.

In another aspect the invention provides methods of treatement of a mutant IDH cancer, comprising adminstering Compound (I) or a pharmaceutically acceptable salt, polymorph, hydrate, solvate or ester thereof, where the cancer is selected from acute myeloid leukemia, colorectal carcinoma, leukemia, lung cancer, lymphoma, multiple myeloma, ovarian cancer, pancreatic cancer and renal cell carcinoma.

In another aspect, the present invention provides the use of Compound (I), or a pharmaceutically acceptable salt, polymorph, hydrate, solvate or ester thereof, for the treatment of cancer bearing an IDH mutation, which cancer is selected from acute myeloid leukemia, breast cancer, colorectal carcinoma, gastric cancer, gliosarcoma, head & neck cancer, hepatocellular carcinoma, leukemia, lung cancer, lymphoma, multiple myeloma, neuroblastoma, ovarian cancer, pancreatic cancer, prostate cancer, renal cell carcinoma, and sarcoma.

In another aspect, the present invention provides the use of Compound (I), or a pharmaceutically acceptable salt, polymorph, hydrate, solvate or ester thereof, for the treatment of cancer bearing an IDH mutation, which cancer is selected from acute myeloid leukemia, brain cancer, breast cancer, colorectal carcinoma, gastric cancer, gliosarcoma, head & neck cancer, hepatocellular carcinoma, leukemia, lung cancer, lymphoma, multiple myeloma, neuroblastoma, ovarian cancer, pancreatic cancer, prostate cancer, renal cell carcinoma, and sarcoma.

In another aspect the invention provides methods for the treatment of cancer bearing an IDH mutation comprising adminstering Compound (I) or a pharmaceutically acceptable salt, polymorph, hydrate, solvate or ester thereof, where the cancer is selected from acute myeloid leukemia, breast cancer, colorectal carcinoma, gastric cancer, gliosarcoma, head & neck cancer, hepatocellular carcinoma, leukemia, lung cancer, lymphoma, multiple myeloma, neuroblastoma, ovarian cancer, pancreatic cancer, prostate cancer, renal cell carcinoma, and sarcoma.

In another aspect, the present invention provides the use of Compound (I), or a pharmaceutically acceptable salt, polymorph, hydrate, solvate or ester thereof, for the treatment of a mutant IDH cancer, which cancer is selected from acute T-cell lymphoblastic leukemia, acute promyelocytic leukemia, acute myeloid leukemia, anaplastic large cell lymphoma, biphenotypic B myelomonocytic leukemia, B-cell lymphoma, breast cancer, Burkitt lymphoma, chronic myeloid leukemia, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma, sarcoma and T-cell lymphoma.

In another aspect the invention provides methods of treatment of mutant IDH cancer, comprising adminstering Compound (I) or a pharmaceutically acceptable salt, polymorph, hydrate, solvate or ester thereof, where the cancer is selected from acute T-cell lymphoblastic leukemia, acute promyelocytic leukemia, acute myeloid leukemia, anaplastic large cell lymphoma, biphenotypic B myelomonocytic leukemia, B-cell lymphoma, breast cancer, Burkitt lymphoma, chronic myeloid leukemia, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma, sarcoma and T-cell lymphoma.

In another aspect, the present invention provides methods of treating mutant IDH cancer, which cancer is selected from lung cancer, leukemia, acute myeloid leukemia, gliosarcoma, colorectal carcinoma, head & neck cancer, hepatocellular carcinoma, multiple myeloma, lymphoma, breast cancer, neuroblastoma, ovarian cancer, gastric cancer, pancreatic cancer, prostate cancer, renal cell carcinoma, and sarcoma.

In another aspect, the present invention provides the use of Compound (I), or a pharmaceutically acceptable salt, polymorph, hydrate, solvate or ester thereof, for the treatment of cancer, which cancer is selected from lung cancer, leukemia, acute myeloid leukemia, gliosarcoma, colorectal carcinoma, head & neck cancer, hepatocellular carcinoma, multiple myeloma, lymphoma, breast cancer, neuroblastoma, ovarian cancer, gastric cancer, pancreatic cancer, prostate cancer, renal cell carcinoma, and sarcoma.

In another aspect, the present invention provides the use of Compound (I), or a pharmaceutically acceptable salt, polymorph, hydrate, solvate or ester thereof, for the treatment of mutant IDH cancer, which cancer is selected from leukemias, lymphomas, sarcomas and solid tumors.

In another aspect the invention provides methods for the treatment of mutant IDH cancer, comprising adminstering Compound (I) or a pharmaceutically acceptable salt, polymorph, hydrate, solvate or ester thereof, wherein the cancer is selected from leukemias, lymphomas, sarcomas and solid tumors.

In another aspect, the present invention provides methods for use of Compound (I), or a pharmaceutically acceptable salt, polymorph, hydrate, solvate or ester thereof, for the treatment of mutant IDH cancer, and methods of treating cancer, which cancer is selected from colorectal cancer, leukemia and lymphoma. In another aspect the invention provides methods for the treatment of mutant IDH cancer comprising administering Compound (I) or a pharmaceutically acceptable salt, polymorph, hydrate, solvate or ester thereof, wherein the cancer is selected from colorectal cancer, leukemia and lymphoma.

In another aspect, the present invention provides the use of Compound (I) of the present invention, or a pharmaceutically acceptable salt, polymorph, hydrate, solvate or ester thereof, for the treatment of mutant IDH cancer and methods of treating mutant IDH cancer, which cancer is selected from colorectal cancer, leukemia and lymphoma.

In another aspect, the present invention provides the use of Compound (I), or a pharmaceutically acceptable salt, polymorph, hydrate, solvate or ester thereof, for the treatment of one or more cancer types potentially bearing an IDH mutation and methods of treating one or more cancer types potentially bearing an IDH mutation, where cancer is selected from ALL, AML, APL, CMML, DLBCL, MDS, MCL, T-NHL, colorectal cancer, melanoma and ovarian cancer.

In another aspect the invention provides methods for the treatment of mutant IDH cancer, comprising administering Compound (I) or a pharmaceutically acceptable salt, polymorph, hydrate, solvate or ester thereof, where the cancer is selected from ALL, AML, APL, CMML, DLBCL, MDS, MCL, T-NHL, colorectal cancer, melanoma and ovarian cancer.

In another aspect, the present invention provides the use of Compound (I) of the present invention, or a pharmaceutically acceptable salt, polymorph, metabolite, hydrate, solvate or ester thereof, for the treatment of one or more cancer types potentially bearing an IDH mutation, where cancer is selected from
leukemias including but not limited to acute lymphoblastic leukemia (ALL), acute myeloid leukemia (AML), acute T-cell leukemia, acute monocytic leukemia, acute promyelocytic leukemia (APL), bisphenotypic B myelomonocytic leukemia, chronic myelogenous leukemia, chronic myeloid leukemia, chronic myelomonocytic leukemia (CMML), large granular lymphocytic leukemia, and myelodysplastic syndrome (MDS), which can develop into an acute myeloid leukemia,
lymphomas including but not limited to AIDS-related lymphoma, chronic lymphocytic lymphoma, non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, B-cell lymphoma, cutaneous T-cell lymphoma, Burkitt lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma;
sarcomas including but not limited to sarcoma of the soft tissue, gliosarcoma, osteosarcoma, malignant fibrous histiocytoma, lymphosarcoma, and rhabdomyosarcoma;
and
solid tumors including but not limited to breast cancer, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma and sarcoma.

In another aspect the invention provides method for the treatment of cancer, potentially bearing an IDH mutation, comprising administering an effective amount of Compound (I) where the cancer is selected from
leukemias including but not limited to acute lymphoblastic leukemia (ALL), acute myeloid leukemia (AML), acute T-cell leukemia, acute monocytic leukemia, acute promyelocytic leukemia, bisphenotypic B myelomonocytic leukemia, chronic myelogenous leukemia, chronic myeloid leukemia, chronic myelomonocytic leukemia (CMML), large granular lymphocytic leukemia, and myelodysplastic syndrome (MDS), which can develop into an acute myeloid leukemia,
lymphomas including but not limited to AIDS-related lymphoma, chronic lymphocytic lymphoma, non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, B-cell lymphoma, cutaneous T-cell lymphoma, Burkitt lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma;
sarcomas including but not limited to sarcoma of the soft tissue, gliosarcoma, osteosarcoma, malignant fibrous histiocytoma, lymphosarcoma, and rhabdomyosarcoma;
and
solid tumors including but not limited to breast cancer, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma and sarcoma.

In another aspect, the present invention provides a method for inhibiting cell proliferation or viability in a cancer cell, the method comprising contacting the cell bearing an IDH mutation with Compound (I), thereby inhibiting cell proliferation or viability.

In a further aspect the present invention provides a method for inhibiting Dihydroorotate Dehydrogenase (DHODH) enzymatic activity, the method comprising contacting DHODH with Compound (I), thereby inhibiting DHODH enzymatic activity and inhibiting mutated isocitrate dehydrogenase (mIDH) enzymatic activity comprising contacting mIDH with Compound (I), thereby inhibiting mIDH enzymatic activity.

In yet a further aspect the present invention provides a method for treating lymphoma mentioned above in a subject, wherein the lymphoma is potentially bearing an IDH mutation and is selected from the group AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, B-cell lymphoma, cutaneous T-cell lymphoma, Burkitt lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma.

In another aspect, the present invention provides a method for treating leukemia potentially bearing an IDH mutation in a subject, the method comprising administering to the subject an effective amount of Compound (I) thereby treating the leukemia.

In yet a further aspect the present invention provides a method for treating leukemia potentially bearing an IDH mutation mentioned above, wherein the leukemia is selected from the group acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.

If it is stated “preventing or treating” or “treatment or prophylaxis” or the like, treating/treatment is preferred.

The term “treating” or “treatment” as stated throughout this document is used conventionally, for example the management or care of a subject for the purpose of combating, alleviating, reducing, relieving, improving the condition of a disease or disorder, such as a carcinoma.

The methods as described supra comprising use of Compound (I) can be used in particular in therapy and prevention, i.e. prophylaxis, of tumour growth and metastases, especially in solid tumours of all indications and stages with or without pre-treatment of the tumour growth.

In addition, the methods as described supra comprising use of Compound (I) can also be used in combination with radiotherapy and/or surgical intervention.

In a further embodiment of the present invention, Compound (I) may be used to sensitize a cell to radiation, i.e. treatment of a cell potentially bearing an IDH mutation with Compound (I) prior to radiation treatment of the cell renders the cell more susceptible to DNA damage and cell death than the cell would be in the absence of any treatment with a compound of the present invention. In one aspect, the cell is treated with Compound (I) of the present invention.

Thus, the present invention also provides a method of killing a cell, wherein Compound (I) is administered to a cell bearing an IDH mutation in combination with conventional radiation therapy.

The present invention also provides a method of rendering a cell potentially bearing an IDH mutation more susceptible to cell death, wherein the cell is treated with Compound (I) prior to the treatment of the cell to cause or induce cell death. In one aspect, after the cell is treated with Compound (I) of the present invention, the cell is treated with at least one compound, or at least one method, or a combination thereof, in order to cause DNA damage for the purpose of inhibiting the function of the normal cell or killing the cell.

In other embodiments the invention relates to a method for the treatment of mutant IDH cancer, comprising contacting a cell potentially bearing an IDH mutation with at least one DNA damaging agent and Compound (I), thereby treating the cancer.

In one embodiment, the DNA damaging agent contacts the cell potentially bearing an IDH mutation prior to, during, or concurrently with Compound (I).

In one embodiment, the DNA damaging agent contacts the cell potentially bearing an IDH mutation after Compound (I).

In another embodiment, contacting the cell with Compound (I) sensitizes the cell potentially bearing an IDH mutation to cell death.

In other embodiments of the present invention, a cell is killed by treating the cell potentially bearing an IDH mutation with at least one DNA damaging agent, i.e. after treating a cell with Compound (I) to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell potentially bearing an IDH mutation with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, Compound (I) is administered to a cell potentially bearing an IDH mutation prior to the radiation or other induction of DNA damage in the cell.

Thus in other embodiments of the invention the invention relates to a method for the treatment of mutant IDH cancer, in a subject comprising administering to the subject an effective amount of Compound (I) prior to, during, or concurrently with exposing the subject to radiation and/or administration of a DNA damaging agent.

Thus in other embodiments of the invention the invention relates to a method for the treatment of mutant IDH cancer in a subject comprising administering to the subject an effective amount of Compound (I) after having exposed the subject to radiation and/or administration of a DNA damaging agent.

In another aspect of the invention, Compound (I) of the present invention is administered to a cell bearing an IDH mutation concomitantly with the radiation or other induction of DNA damage in the cell.

In yet another aspect of the invention, Compound (I) is administered to a cell bearing an IDH mutation immediately after radiation or other induction of DNA damage in the cell has begun.

In another aspect, the cell bearing an IDH mutation is in vitro. In another embodiment, the cell bearing an IDH mutation is in vivo.

In accordance with a further aspect, the present invention provides Compound (I), as or tautomers, N-oxides, and salts thereof, or salts of tautomers or N-oxides, particularly pharmaceutically acceptable salts thereof, or mixtures of same, for use in the treatment or prophylaxis of diseases, in particular hyperproliferative and/or inflammatory disorders, bearing an IDH mutation.

In accordance with a further aspect, the present invention provides Compound (I), as described supra, or tautomers, N-oxides, and salts thereof, or salts of tautomers or N-oxides, particularly pharmaceutically acceptable salts thereof, or mixtures of same, for use in the treatment or prophylaxis of diseases, in particular inflammatory disorders bearing an IDH mutation.

In accordance with a further aspect, the present invention provides Compound (I), as described supra, or tautomers, N-oxides, and salts thereof, or salts of tautomers or N-oxides, for use in the treatment or prophylaxis of diseases, in particular hyperproliferative disorders, particularly benign hyperproliferative disorders, more particularly cancer bearing an IDH mutation.

Another aspect is that Compound (I) additionally may be active as inhibitors of cell proliferation of cells bearing an IDH mutation.

A further aspect is that the activity of Compound (I) may be determined by detecting the reaction product of the new reaction being catalyzed by mutated IDH which is 2-hydroxyglutarate, more specifically R(−)-2-hydroxyglutarate.

In accordance with another aspect, the invention provides methods of treatment of cancer comprising administering to a subject in need thereof a therapeutically effective amount of Compound (I) thereby inhibiting DHODH as well as inhibiting cell proliferation of cells comprising an IDH mutation.

In accordance with a further aspect, the present invention provides a method of treatment of cancer with Compound (I) comprising the step of determining IDH mutation in a tumor cell.

In accordance of another aspect, the present invention provides a method of treatment of cancer comprising the steps of determining IDH mutation in a tumor cell, further comprising determining the status of proliferation when contacting said tumor cell with Compound (I).

In accordance of another aspect, the present invention provides a method of treatment of cancer comprising the steps of determining IDH mutation in a tumor cell, further comprising determining the status of proliferation when contacting said tumor cell with the DHODH inhibitor Compound (I) further comprising administering Compound (I) to the subject in need thereof.

In accordance with a further aspect, the present invention provides use of Compound (I), as described supra, or a tautomer, an N-oxide, and a salt thereof, or a salt of a tautomer or an N-oxide, particularly a pharmaceutically acceptable salt thereof, or a mixture of same, for the preparation of a pharmaceutical composition, preferably a medicament, for the prophylaxis or treatment of diseases, in particular hyperproliferative disorders, particularly benign hyperproliferative disorders, more particularly cancer bearing an IDH mutation.

In accordance with a further aspect, the present invention provides a method for the treatment or prophylaxis of diseases, in particular hyperproliferative and/or inflammatory disorders bearing an IDH mutation, comprising administering to a subject in need thereof an effective amount of Compound (I), as described supra, or a tautomer, an N-oxide, and a salt thereof, or a salt of a tautomer or an N-oxide, particularly a pharmaceutically acceptable salt thereof, or a mixture of same.

In accordance with a further aspect, the present invention provides a method for the treatment or prophylaxis of diseases, in particular hyperproliferative disorders, particularly benign hyperproliferative disorders, more particularly cancer bearing an IDH mutation, comprising administering to a subject in need thereof an effective amount of Compound (I), as described supra, or a tautomer, an N-oxide, and a salt thereof, or a salt of a tautomer or an N-oxide, particularly a pharmaceutically acceptable salt thereof, or a mixture of same.

In accordance with a further aspect, the present invention provides pharmaceutical compositions, in particular a medicament, comprising Compound (I), as described supra, or a tautomer, an N-oxide, and a salt thereof, or a salt of a tautomer or an N-oxide, particularly a pharmaceutically acceptable salt, or a mixture of same, and one or more excipients, in particular one or more pharmaceutically acceptable excipient(s) for use in the treatment of cancer bearing an IDH mutation. Conventional procedures for preparing such pharmaceutical compositions in appropriate dosage forms can be utilized.

The present invention furthermore provides pharmaceutical compositions, in particular medicaments, which comprise Compound (I), conventionally together with one or more pharmaceutically suitable excipients, and to their use for the above mentioned purposes.

It is possible for Compound (I) to have systemic and/or local activity. For this purpose, Compound (I) can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for Compound (I) to be administered in suitable administration forms.

For oral administration, it is possible to formulate Compound (I) to dosage forms known in the art that deliver Compound (I) rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the Compound (I) according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

Compound (I) can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

In accordance with another aspect, the present invention provides pharmaceutical combinations, in particular medicaments, comprising Compound (I) and at least one or more further active ingredients, in particular for the treatment and/or prophylaxis of a hyperproliferative disorder, particularly cancer bearing an IDH mutation.

Particularly, the present invention provides a pharmaceutical combination, which comprises:
one or more first active ingredients, in particular at least Compound (I) as defined supra, and
one or more further active ingredients, in particular anti-cancer agents.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as Compound (I), and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

Compound can be administered as the sole pharmaceutical agent or in combination with one or more other pharmaceutically active ingredients where the combination causes no unacceptable adverse effects. The present invention also provides such pharmaceutical combinations. For example, the compound (I) can be combined with known anti-cancer agents.

Examples of anti-cancer agents include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporf in, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Furthermore Compound (I) may be adminstered in combination with a mutant IDH inhibitor.

Based upon standard laboratory techniques known to evaluate compounds useful for the treatment of hyperproliferative and/or inflammatory disorders, by standard toxicity tests and by standard pharmacological assays for the determination of treatment of the conditions identified above in mammals, and by comparison of these results with the results of known active ingredients or medicaments that are used to treat these conditions, the effective dosage of Compound (I) can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg (e.g. about 0.5 mg to about 5 mg, about 5 mg to about 50 mg, about 50 mg to about 500 mg, about 500 mg to about 1500 mg, etc.) of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

In other embodiments of the invention the total amount of the active ingredient to be administered will generally range from 0.001 mg/kg to 200 mg/kg body weight per day, and preferably from 0.01 mg/kg to 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from 0.5 mg to 1500 mg of active ingredient, and can be administered one or more times per day or less than once a day.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Experimental Section

NMR peak forms are stated as they appear in the spectra, possible higher order effects have not been considered.

The ¹H-NMR data of selected examples are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “Minimum Height”<1%.

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations

| Abbreviation | Meaning |
|---|---|
| aq. | aqueous |
| ACN | acetonitrile |
| br | broad (¹H-NMR signal) |
| cat. | catalytic |
| CDI | 1,1′-carbonyldiimidazole |
| CI | chemical ionisation |
| conc. | concentrated |
| d | doublet |
| DAD | diode array detector |
| DCM | dichloromethane |
| dd | double-doublet |
| ddd | double-doublet-doublet |
| DIPEA | diisopropylethylamine |
| DMAP | 4-dimethylaminopyridine |
| DMF | N,N-dimethylformamide |
| DMSO | dimethylsulfoxide |
| eq. | equivalent |
| ESI | electrospray (ES) ionisation |
| h | hour(s) |
| HATU | 1-[bis(dimethylamino)methylene]-1H-1,2,3-triazolo[4,5- |
| | b]pyridinium 3-oxid hexafluorophosphate |
| HBTU | (o-benzotriazole-10-yl)-N,N,N′,N,-tetramethyluronium |
| | hexafluorophosphate |
| HCl | hydrochloric acid |
| HPLC | high performance liquid chromatography |
| LC-MS | liquid chromatography mass spectrometry |
| m | multiplet |
| min | minute(s) |
| MeOH | methanol |
| Mp. | melting point |
| MS | mass spectrometry |
| NMR | nuclear magnetic resonance spectroscopy: chemical |
| | shifts (δ) are given in ppm. The chemical shifts were |
| | corrected by setting the DMSO signal to 2.50 ppm |
| | unless otherwise stated. |
| MTP | microtiter plate |
| q | quartet |
| quin | quintet |
| r.t. or rt or RT | room temperature |
| Rt | retention time (as measured either with HPLC or UPLC) |
| | in minutes |
| s | singlet |
| sxt | sextet |
| sep | septet |
| t | triplet |
| td | triple-doublet |
| THF | tetrahydrofuran |
| TLC | thin-layer chromatography |
| UPLC | ultra performance liquid chromatography |

Other abbreviations have their meanings customary per se to the skilled person.

The various aspects and embodiments of the invention described in this application are illustrated by the following examples which are not meant to limit the invention in any way.

Experimental Section—General Part

All reagents, for which the synthesis is not described in the experimental part, are either commercially available, or are known compounds or may be formed from known compounds by known methods by a person skilled in the art.

The compounds and intermediates produced according to the methods disclosed herein may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

UPLC-MS Standard Procedures

Analytical UPLC-MS was performed as described below. The masses (m/z) are reported from the positive mode electrospray ionisation unless the negative mode is indicated (ESI−).

Method A (HPLC-MS):

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

Method B (HPLC-MS):

Instrument: Waters Acquity UPLCMS SingleQuad; Column: Acquity UPLC BEH C18 1.7 μm, 50×2.1 mm; eluent A: water+0.2 Vol-% aq. ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

Experimental Section—Intermediates

Intermediate 1

### 4-ethyl-5-(hydroxymethyl)-2,4-dihydro-3H-1,2,4-triazol-3-one

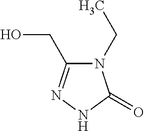

2-hydroxyacetohydrazide (50.0 g, 555 mmol) was dissolved in water (125 mL) and cooled to 0° C. Ethyl isocyanate (44 ml, 560 mmol) was added (very exotherm!) and the resulting mixture was stirred at room temperature overnight. The resulting suspension was treated with sodium hydroxide solution (64 g, 50 wt % in water) (exotherm). The resulting solution was heated to 95° C. overnight. The yellow reaction mixture was neutralized with concentrated hydrochloric acid and the resulting cloudy solution was concentrated to dryness. The solids were triturated with a mixture of dichloromethane and isopropanol (4:1, 750 mL), the solution was filtered off and concentrated to dryness again to yield ˜78 g of crude product. The product was recrystallized from ethyl acetate to yield the desired product (56.2 g, 71% yield).

¹H-NMR (400 MHz, DMSO-d6) δ [ppm]: 1.156 (6.88), 1.174 (16.00), 1.192 (7.02), 2.518 (0.48), 3.606 (2.02), 3.623 (6.84), 3.641 (6.79), 3.659 (1.96), 4.316 (4.51), 5.540 (0.94).

Intermediate 2

### 4-bromo-5-fluoro-2-{[(2S)-1,1,1-trifluoropropan-2-yl]oxy}benzonitrile

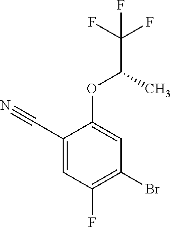 [SMILES: C[C@H](Oc1cc(Br)c(F)cc1C#N)C(F)(F)F]

To a stirred suspension of 4-bromo-2,5-difluorobenzonitrile (91 g, 417 mmol) and potassium carbonate (173 g, 1.25 mol) in N,N-dimethylformamide (910 ml) was added (S)-1,1,1-trifluoropropanol [CAS 3539-97-7] dropwise (52.4 g, 460 mmol). The resulting mixture was heated at 70° C. for for 15 hours and cooled to room temperature. The reaction was concentrated and the residue was diluted with water. The aqueous solution was extracted with DCM (3×). The combined organic washes were washed with brine, dried over sodium sulfate, filtered, and concentrated under reduced pressure to give an oil (136.6 g). The residue was triturated with a mixture of hexanes and toluene (9:1, 200 mL) to give the desired product as a white solid (90.3 g, 93% purity, 64% yield).

LC-MS (Method A): Rₜ=1.29 min; MS (ESIpos): m/z=312.0 [M+H]⁺.

¹H-NMR (400 MHz, CHLOROFORM-d) δ [ppm]: 1.511 (4.41), 1.531 (15.64), 1.533 (16.00), 1.548 (15.84), 1.550 (15.83), 4.566 (1.08), 4.581 (2.68), 4.597 (3.18), 4.612 (2.62), 4.626 (1.01), 7.194 (5.27), 7.207 (7.60), 7.220 (7.57).

Intermediate 3

### 4-bromo-5-fluoro-2-{[(2S)-1,1,1-trifluoropropan-2-yl]oxy}benzoic acid

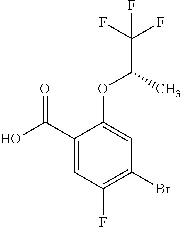 [SMILES: C[C@H](Oc1cc(Br)c(F)cc1C(=O)O)C(F)(F)F]

To a solution of 4-bromo-5-fluoro-2-{[(2S)-1,1,1-trifluoropropan-2-yl]oxy}benzonitrile (Intermediate 2, 27.4 g, 87.9 mmol) in ethanol (90 ml) was added aqueous sodium hydroxide (2 N, 140 ml) and the resulting mixture was heated to 90° C. for 20 hours. The resulting solution was cooled to room temperature, diluted with water, and extracted with dichloromethane. The aqueous phase was acidified with 2 N aqueous hydrochloric acid (pH 2) upon which a white solid precipitated. The suspension was stirred for further 15 minutes, the solid was filtered off, washed with water and dried in vacuo. to yield an off-white solid (25.97 g, 89%), which was used for the next step without further purification.

LC-MS (Method A): Rₜ=1.16 min; MS (ESIpos): m/z=331 [M+H]⁺.

¹H-NMR (400 MHz, DMSO-d6) δ [ppm]: 1.396 (15.96), 1.411 (16.00), 1.469 (0.69), 1.484 (0.45), 2.518 (3.48), 2.523 (2.36), 5.288 (1.19), 5.304 (2.89), 5.320 (3.73), 5.336 (2.70), 5.352 (1.05), 7.612 (11.35), 7.634 (11.12), 7.743 (7.88), 7.757 (7.91).

Intermediate 4

### 4-bromo-5-fluoro-2-{[(2S)-1,1,1-trifluoropropan-2-yl]oxy}benzoyl chloride

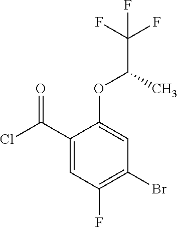 [SMILES: C[C@H](Oc1cc(Br)c(F)cc1C(=O)Cl)C(F)(F)F]

To a solution of 4-bromo-5-fluoro-2-{[(2S)-1,1,1-trifluoropropan-2-yl]oxy}benzoic acid (Intermediate 3, 15.0 g, 45.3 mmol) in dichloromethane (230 ml) was added N,N-dimethylformamide (350 μl), followed by dropwise addition of ethanedioyl dichloride (4.7 ml, 54 mmol). The reaction mixture was stirred at room temperature for one hour, and concentrated under reduced pressure. The title compound was obtained as brown oil (15.84 g, quantitative), which was used for the next step without purification. For analytic, a small amount of the product was treated with methanol, to yield the corresponding methyl ester, which was detected by LC-MS.

LC-MS (Method A) [methyl ester]: Rₜ=1.36 min; MS (ESIpos): m/z=345 [M+H]⁺.

Intermediate 5

### 4-bromo-N-(2-chloro-6-fluorophenyl)-5-fluoro-2-{[(2S)-1,1,1-trifluoropropan-2-yl]oxy}benzamide

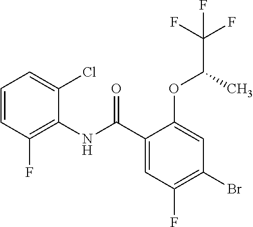 [SMILES: C[C@H](Oc1cc(Br)c(F)cc1C(=O)Nc1c(F)cccc1Cl)C(F)(F)F]

4-Bromo-5-fluoro-2-{[(2S)-1,1,1-trifluoropropan-2-yl]oxy}benzoyl chloride (Intermediate 4, 31.9 g, 91.3 mmol) was dissolved in DCM (300 mL) and added to a solution of 2-chloro-6-fluoroaniline (14.6 g, 100.4 mmol) and triethylamine (14 ml, 100 mmol) in DCM (400 mL). The mixture was stirred at room temperature for 30 min. The mixture was concentrated to yield 47.9 g of crude product. The crude product was dissolved in ethanol (250 mL) and water (500 mL) was added slowly. The resulting precipitate was filtered off, the solids were washed with water and dried to yield the desired product (39.3 g, 84.5% yield).

Synthesized analogously to Intermediate 70 from Intermediate 68 and 2-chloro-6-fluoroaniline.

LC-MS (Method A): Rₜ=1.44 min; MS (ESIpos): m/z=458 [M+H]⁺.

¹H-NMR (400 MHz, DMSO-d6) δ [ppm]: 1.343 (1.46), 1.359 (1.66), 1.387 (0.50), 1.437 (16.00), 1.452 (15.88), 1.475 (0.73), 1.495 (0.82), 1.513 (0.53), 1.907 (0.67), 2.332 (1.23), 2.518 (7.59), 2.523 (4.64), 2.673 (1.20), 5.400 (1.28), 5.416 (2.77), 5.432 (3.47), 5.448 (2.51), 5.464 (1.02), 7.323 (1.55), 7.328 (1.69), 7.347 (3.94), 7.354 (2.01), 7.363 (2.31), 7.371 (2.98), 7.383 (2.19), 7.395 (1.78), 7.402 (3.88), 7.416 (5.31), 7.425 (9.08), 7.431 (9.78), 7.445 (2.16), 7.481 (0.67), 7.502 (0.44), 7.528 (6.83), 7.549 (6.80), 7.665 (0.44), 7.680 (0.70), 7.694 (0.41), 7.820 (5.90), 7.833 (5.87), 9.977 (11.65).

### EXAMPLE 1

### N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide

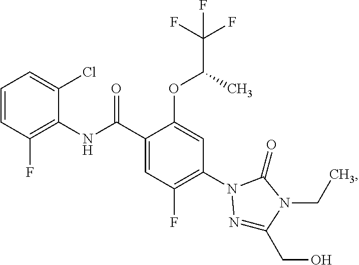 [SMILES: CCn1c(CO)nn(-c2cc(O[C@@H](C)C(F)(F)F)c(C(=O)Nc3c(F)cccc3Cl)cc2F)c1=O]

4-Bromo-N-(2-chloro-6-fluorophenyl)-5-fluoro-2-{[(2S)-1,1,1-trifluoropropan-2-yl]oxy}benzamide (Intermediate 5, 100 mg, 218 μmol), 4-ethyl-5-(hydroxymethyl)-2,4-dihydro-3H-1,2,4-triazol-3-one (Intermediate 1, 46.8 mg, 327 μmol), tris(dibenzylideneacetone)dipalladium(0) (20 mg, 22 μmol), 4,5-bis(diphenylphosphino)-9,9-dimethylxanthene (38 mg, 65 μmol), and cesium carbonate (142 mg, 436 μmol) were loaded into a microwave vial. The vial was purged with argon, dioxane (2 mL, degassed) was added, and the vial was sealed. The mixture was stirred for 17 h at 110° C. The resulting suspension was filtered over Celite, washed with ethyl acetate and concentrated. Mass triggered preparative chromatography yielded the desired product (47.0 mg, 40% yield).

LC-MS (Method A): Rₜ=1.16 min; MS (ESIpos): m/z=521 [M+H]⁺.

¹H-NMR (400 MHz, DMSO-d6) δ [ppm]: 1.137 (0.43), 1.233 (0.49), 1.264 (6.16), 1.282 (13.73), 1.300 (6.05), 1.436 (9.08), 1.451 (8.92), 2.084 (8.00), 2.322 (2.38), 2.326 (3.08), 2.331 (2.22), 2.518 (16.00), 2.522 (10.27), 2.664 (2.43), 2.669 (3.14), 2.673 (2.27), 3.764 (1.73), 3.782 (5.24), 3.800 (5.08), 3.818 (1.51), 4.479 (8.00), 4.493 (8.00), 5.331 (0.59), 5.347 (1.41), 5.363 (1.78), 5.380 (1.24), 5.396 (0.49), 5.759 (1.24), 5.789 (2.65), 5.803 (5.89), 5.817 (2.38), 7.337 (0.97), 7.356 (2.16), 7.372 (1.35), 7.380 (1.62), 7.388 (1.14), 7.408 (2.00), 7.422 (2.43), 7.433 (4.54), 7.439 (5.08), 7.453 (1.14), 7.550 (3.41), 7.575 (3.57), 7.588 (3.19), 7.602 (2.92), 10.065 (7.03).

Experimental Section—Biological Assays

The following table 2 lists the abbreviations used herein, in particular in the Biological Assys part of the Experimental Section:

TABLE 2
Abbreviations

| | |
|---|---|
| ATCC | American Type Culture Collection |
| DDK | Name of a polypeptide tag |
| DCM | dichloromethane |
| DHODH | Dihydroorotate Dehydrogenase |
| DMSO | dimethylsulfoxide |
| h | hour(s) |
| IC₅₀ | half maximal inhibitory concentration |
| μM | micromolar |
| mM | millimolar |
| MTP | Microtiter plate |
| MYC | name of a polypeptide tag |
| μl | microliter |
| nM | nanomolar |
| PBS | Phosphate Buffered Saline |
| RPMI | Roswell Park Memorial Institute |
| rt | room temperature |
| THP | cell line name |
| Triton X | name of a detergent |
| Tris | tris(hydroxymethyl)aminomethane |

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

The in vitro activity of Compound (I) can be demonstrated in the following assays:

In Vitro Assay 1: DHODH Enzymatic Assay—1

The enzymatic assay couples DHODH activity with bleaching of the dye 2,6-dichlorophenolindophenol (DCIP) (Knecht and Loffler, 1998; Miller et al., 1968). The assay was conducted in buffer containing 50 mM Tris, 0.1% Triton X-100, 150 mM potassium chloride, 2 nM DHODH, 1 mM dihydroorotate, 0.1 mM decylubiquinone, 0.06 mM DCIP, and 2% DMSO at pH 8.0 at 32 degree celsius. The reaction was initiated by addition of substrates. Enzyme activity was monitored kinetically by the reduction in DCIP absorbance at 600 nm. Purified recombinant human DHODH enzyme was purchased from Novus (cat. no. NBP1-98916). Other chemicals were purchased from Sigma-Aldrich. Absorbance measurements were obtained using a BMG clarion star plate-reading spectrophotometer.

In Vitro Assay 2: THP-1 Proliferation Assay—1 (AML)

2000 cells/well of THP-1 cells were seeded in RPMI 1640 with Glutamax (Gibco, #11875-093) and 10% fetal calf serum (Biochrom, #S0615) in 384-well plates. The next day, cells were incubated with different concentrations of test compounds for 72 h. Cellular viability was analyzed using CellTiter-Glo® Luminescent Cell Viability Assay (Promega, #G7570) according to manufacturer's instructions.

TABLE 3
IC₅₀ values Compound (I) and reference
compound in in vitro assays 1-2

| | In vitro Assay 1: | In vitro Assay 2: |
|---|---|---|
| | DHODH enzymatic assay 1 | THP-1 proliferation |
| Example No | IC₅₀ [mol/l] (mean values) | assay (AML) IC₅₀ [mol/l] |
| Compound (I) | 4.17E−09 | 4.25E−09 |
| Ref. Cpd. 1 | 3.00E−05 | |

Assay 3—Cell Proliferation Panel Assay

Cells were grown in RPMI 1640, 10% FBS, 2 mM L-alanyl-L-glutamine, 1 mM Na pyruvate or a special medium. Cells were seeded into 384-well plates and incubated in a humidified atmosphere of 5% CO2 at 37° C. Compounds were added the day following cell seeding. At the same time, a time zero untreated cell plate was generated. After a 3-day incubation period, cells were fixed and stained to allow fluorescence imaging of nuclei.

Compounds were serially diluted in half-log steps from the highest test concentration 10 μM, and assayed over 10 concentrations with a maximum assay concentration of 0.1% DMSO. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software.

Data Analysis

Cell proliferation was measured by the fluorescence intensity of an incorporated nuclear dye. The output is referred to as the relative cell count, where the measured nuclear intensity is transformed to percent of control (POC) using the following formula:

$$POC = \frac{{I}_{x}}{{I}_{0}} \times 100$$

Where Iₓ is the nuclear intensity at concentration x, and I₀ is the average nuclear intensity of the untreated vehicle wells.

Cellular response parameters were calculated using nonlinear regression to a sigmoidal single-site dose response model:

$$y = A + \frac{B - A}{1 + {(}^{C} {\text{/}}_{x} )}$$

Where y is a response measured at concentration x, A and B are the lower and upper limits of the response, C is the concentration at the response midpoint (EC₅₀), and D is the Hill Slope (Fallahi-Sichani, M., S. Honardejad, L. M. Heiser, J. W. Gray, and P. K. Sorger (2013). Metrics other than potency reveal systematic variation in responses to cancer drugs. Nat. Chem. Biol. 9: 708-714.).

Time zero non-treated plates were used to determine the number of doublings during the assay period, using the formula:

$$Doublings = {log}_{ 2} ( \frac{N}{{N}_{T 0}} )$$

Where N is the cell number in untreated wells at the assay end point and N_{T0} is the cell number at the time of compound addition.

Cell count IC₅₀ is the test compound concentration at 50% of maximal possible response. EC₅₀ is the test compound concentration at the curve inflection point or half the effective response (parameter C of the fitted curve solution).

Curve-fitting, calculations, and report generation was performed using a custom data reduction enaine and MathIQ based software (AIM).

TABLE 2
Data obtained from assay 3

| | | | Cell Count | Cell Count |
|---|---|---|---|---|
| Com- | | | EC50 | IC50 |
| pound | Cell Line | Origin | (microM) | (microM) |
| Example 1 | Colo 320DM | Colorectal | 1.23E−03 | 5.10E−03 |
| | | carcinoma | | |
| Example 1 | 786-O | Renal cell | 8.59E−04 | 2.36E−03 |
| | | carcinoma | | |
| Example 1 | A498 | Renal cell | >1.00E+01 | >1.00E+01 |
| | | carcinoma | | |
| Example 1 | ACHN | Renal cell | 3.12E−03 | 1.83E−02 |
| | | carcinoma | | |
| Example 1 | ARH-77 | Leukemia | 9.20E−04 | 1.39E−03 |
| Example 1 | BC-1 | Lymphoma | 1.02E−03 | 1.59E−03 |
| Example 1 | BV-173 | Leukemia | 9.06E−04 | 7.79E−02 |
| Example 1 | Colo 205 | Colorectal | 8.36E−04 | 1.22E−03 |
| | | carcinoma | | |
| Example 1 | CA46 | Lymphoma | 3.11E−03 | 1.35E−02 |
| Example 1 | CCRFCEM | Leukemia | 9.93E−04 | 1.56E−03 |
| Example 1 | CEM-C1 | Leukemia | 9.10E−04 | 1.28E−03 |
| Example 1 | CFPAC-1 | Pancreatic | 4.55E−03 | 1.64E−02 |
| | | cancer | | |
| Example 1 | CML-T1 | Leukemia | 3.52E−04 | 4.95E−04 |
| Example 1 | CaOV3 | Ovarian | 1.29E−02 | >1.00E+01 |
| | | carcinoma | | |
| Example 1 | DMS114 | Lung cancer | 7.62E−03 | >1.00E+01 |
| Example 1 | Daudi | Lymphoma | 6.13E−04 | 1.02E−03 |
| Example 1 | DLD-1 | Colorectal | 1.06E−03 | 4.01E−03 |
| | | carcinoma | | |
| Example 1 | EM-2 | Leukemia | 1.06E−03 | 1.97E−03 |
| Example 1 | G-401 | Kidney cancer | 2.19E−03 | >1.00E+01 |
| Example 1 | HCT-15 | Colorectal | 9.21E−04 | 2.83E−03 |
| | | carcinoma | | |
| Example 1 | HCT-8 | Colorectal | 1.27E−03 | 2.34E−03 |
| | | carcinoma | | |
| Example 1 | HCT-116 | Colorectal | 8.64E−04 | 1.67E−03 |
| | | carcinoma | | |
| Example 1 | HT-29 | Colorectal | 2.72E−03 | 1.10E−02 |
| | | carcinoma | | |
| Example 1 | JeKo-1 | Lymphoma | 1.79E−03 | 3.01E−03 |
| Example 1 | Jurkat | Leukemia | 6.38E−04 | 9.25E−04 |
| Example 1 | K562 | Leukemia | 1.03E−03 | 1.31E−03 |
| Example 1 | MOLT-3 | Leukemia | 1.14E−03 | 1.49E−03 |
| Example 1 | MOLT-16 | Leukemia | 8.42E−04 | 1.08E−03 |
| Example 1 | MEG01 | Leukemia | 6.60E−04 | 3.05E−03 |
| Example 1 | MHH-PREB-1 | Lymphoma | 7.42E−04 | 8.30E−04 |
| Example 1 | Mia PaCa-2 | Pancreatic | 2.23E−03 | >1.00E+01 |
| | | cancer | | |
| Example 1 | MV-4-11 | Leukemia | 8.14E−04 | 1.20E−03 |
| Example 1 | NAMALWA | Lymphoma | 2.53E−04 | 1.56E−03 |
| Example 1 | NALM-6 | Leukemia | 9.23E−04 | 1.27E−03 |
| Example 1 | PA-1 | Ovarian | 2.07E−03 | 2.88E−03 |
| | | carcinoma | | |
| Example 1 | PANC-1 | Pancreatic | 3.50E−03 | >1.00E+01 |
| | | cancer | | |
| Example 1 | PSN-1 | Pancreatic | 8.57E−04 | 6.46E−03 |
| | | cancer | | |
| Example 1 | Raji | Lymphoma | 8.56E−04 | 1.35E−03 |
| Example 1 | Ramos (RA 1) | Lymphoma | 7.18E−04 | 8.78E−04 |
| Example 1 | RPMI 8226 | Multiple | 2.32E−03 | 4.21E−03 |
| | | Myeloma | | |
| Example 1 | SU-DHL-10 | Lymphoma | 1.26E−03 | 4.09E−03 |
| Example 1 | SHP-77 | Lung cancer | 1.53E−03 | 1.63E−02 |
| Example 1 | SU.86.86 | Pancreatic | 5.08E−03 | >1.00E+01 |
| | | cancer | | |
| Example 1 | U266B1 | Multiple | 4.88E−03 | >1.00E+01 |
| | | Myeloma | | |

Assay 4—Proliferation Assay

TF1/RI32H27 (mIDHJ) and TF1/R140Q11 (mIDH2) cells are plated at 80 k/mL, 90 μl/well and TF1-pLVX (wildtype) cells are pLVX plated at 20 k/ml, 90 μl/well under conditions of RPM1, 10% FBS, G418 and GM-CSF. The test compound can be added on day 0 and Cell Titer-Glo®-assay of Promega is performed on days 3 or 4 and 7. The medium should not be changed during the 7-days culture.

In order to demonstrate a compound's effect on target, uridine and orotate are prepared on concentrations of 8, 40, 200 and 1000 μM and can be added to the cell culture medium separately and a comparative probe without uridine nor orotate should be prepared as well. A suitable single dose of the compound, e.g. 2 μM can be added on day 7. The data obtained can be expressed e.g. as an ATP fold-change: day 3 or day 4 over day 0.

1. A method of treating a mutant IDH cancer in a subject comprising administering to a subject in need thereof a therapeutically effective amount of Compound (I), which N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide 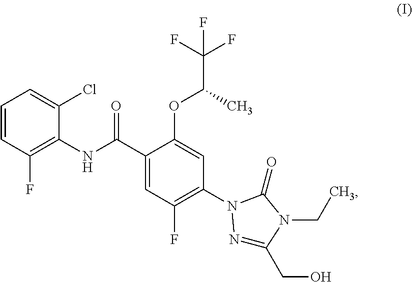 [SMILES: CCn1c(CO)nn(-c2cc(O[C@@H](C)C(F)(F)F)c(C(=O)Nc3c(F)cccc3Cl)cc2F)c1=O]
or a tautomer, an N-oxide, a salt, a salt of a tautomer or a salt of an N oxid N oxide thereof.

2. The method of claim 1 where the mutant IDH cancer is characterized by a mutation in an IDH gene or in an IDH protein.

3. The method of claim 1 further comprising detecting DHO.

4. The method of claim 3 further comprising detecting the presence of a mutant IDH gene or protein.

5. The method of claim 4, wherein the IDH mutant is characterized by a mutation in an IDH-1 gene or in an IDH-1 protein.

6. The method of claim 5, wherein the IDH-1 mutation is an amino acid substitution selected from the group G27D, R132C, R132G, R132H, R132L and R132S.

7. The method of claim 4, wherein the the IDH mutant is characterized by a mutation in an IDH-2 gene or in an IDH-2 protein.

8. The method of claim 7, wherein the IDH-2 mutation is an amino acid substitution selected from the group R140L, R140W, R140Q, R172G and R172K.

9. The method of claim 1, wherein a combination of a mutant IDH inhibitor and Compound (I) is administered.

10. The method of claim 1, wherein the cancer is selected from the group consisting of acute myeloid leukemia, brain cancer, breast cancer, colorectal carcinoma, gastric cancer, gliosarcoma, head & neck cancer, hepatocellular carcinoma, leukemia, lung cancer, lymphoma, multiple myeloma, neuroblastoma, ovarian cancer, pancreatic cancer, prostate cancer, renal cell carcinoma, and sarcoma.

11. The method of claim 10, wherein the lymphoma is selected from the group AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, B-cell lymphoma, cutaneous T-cell lymphoma, Burkitt lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma.

12. The method of claim 10, wherein the leukemia is selected from the group acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.
